(19) United States
(12) Patent Application Publication
(10) Pub. No.: US 2022/0076781 A1
(43) Pub. Date: Mar. 10, 2022

(54) FAST AND STABLE GENOMIC BREEDING VALUE EVALUATING METHOD FOR ANIMAL INDIVIDUALS

(71) Applicants: HUAZHONG AGRICULTURAL UNIVERSITY, Wuhan, Hubei (CN); GUANGZHOU YINGZI TECHNOLOGY CO., LTD., Guangzhou, Guangdong (CN)

(72) Inventors: Shuhong ZHAO, Wuhan, Hubei (CN); Xiaolei LIU, Wuhan, Hubei (CN); Xiang YANG, Guangzhou, Guangdong (CN); Xinyun LI, Wuhan, Hubei (CN); Mengjin ZHU, Wuhan, Hubei (CN); Tao XIANG, Wuhan, Hubei (CN); Yunlong MA, Wuhan, Hubei (CN); Mei YU, Wuhan, Hubei (CN); Zhiquan WANG, Guangzhou, Guangdong (CN); Lilin YIN, Wuhan, Hubei (CN)

(21) Appl. No.: 17/417,007

(22) PCT Filed: Jan. 14, 2019
(86) PCT No.: PCT/CN2019/071514

§ 371 Date: Jun. 21, 2021

(30) Foreign Application Priority Data
Dec. 28, 2018 (CN) 201811620927.X

(51) Int. Cl. G16B 20/20; G16B 40/00; G06F 17/18

(52) U.S. Cl. CPC G16B 20/20; G06F 17/18; G16B 40/00

(57) ABSTRACT

The disclosure provides a fast and stable genomic breeding value evaluating method for animal individuals, and relates to the technical field of animal breeding. In the method, HIBLUP is adopted to perform genomic breeding value prediction using phenotype, genotype and pedigree information, and the final output includes estimated genetic value of individuals, additive effect and dominant effect values of each individual, and back solution values of each genetic marker effect used on genotyping chips. The pedigree, phenotypic and genotypic information can be fully used to predict genetic (additive and dominant) values for each individual animal and as well as effect values for each SNP marker, and the most advanced genomic breeding value prediction and variance component estimation algorithm are realized to realize genomic selection.

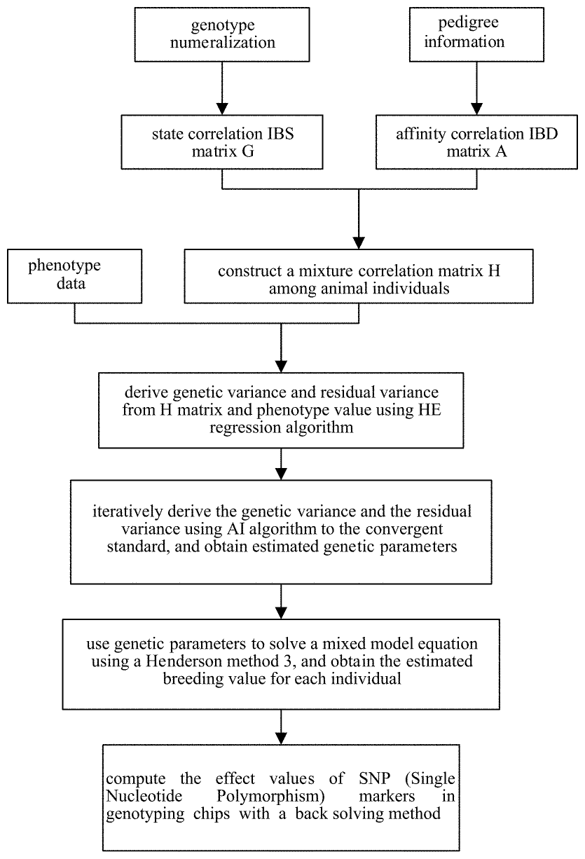

### BACKGROUND OF THE INVENTION

### 1. Field of the Invention

The invention relates to the technical field of animal breeding, in particular to a fast and stable genomic breeding value evaluating method for animal individuals.

### 2. The Prior Arts

With the development of a high-density single-nucleotide polymorphisms (SNPs) genotyping technique covering the whole genome, genomic selection (prediction), as a power tool for genome statistical analysis, is widely applied to predict and estimate genetic value (breeding value) of complex traits in plant and animal breeding, increasingly in human genetics studies. Estimation of variance components is probably the most time consuming part in the process of genomic selection. Previous popular variance component estimation algorithms used in genomic selection, such as EMAI, need iterative computation and the computational complexity of each iteration is very high. Previous genomic selection programs need to compute the inverse matrix of a genomic affinity relationship matrix and the computing time is increasing rapidly with the increased genotyped sample size.

### SUMMARY OF THE INVENTION

The invention aims to solve the technical problem of providing a fast and stable genomic breeding value evaluating method for animal individuals for deficiency existing in the prior art. The HE-AI algorithm based BLUP (namely best linear unbiased prediction) is called “HIBLUP”, HIBLUP makes whole use of pedigree, phenotypic and genotypic information to predict genetic (additive and dominant) effects for each individual animal as well as effect values for each SNP marker, and the most advanced genomic breeding value prediction and variance component estimation algorithm are realized to realize genomic selection.

To solve the above-mentioned technical problem, the technical solution adopted by the invention is a fast and stable genomic breeding value evaluating method for animal individuals, which is characterized in that HIBLUP is adopted to perform genomic breeding value prediction using phenotype, genotype and pedigree information, and a final output includes estimated genetic value of individuals, additive effect and dominant effect values of each individual, and back solution values of each genetic marker effect used on genotyping chips. The fast and stable genomic breeding value evaluating method for animal individuals specifically includes the following steps:

step 1: performing numeralization on genotypes, coding genotypes of AA, AB and BB as 0, 1, and 2, respectively, constructing a relationship A (affinity correlation IBD) matrix and a G (state correlation IBS) matrix among individuals based on pedigree information using a Henderson tabular method and genomic information using a VanRaden method, respectively, and then constructing a mixture correlation matrix H among the animal individuals according to information of both an A matrix and a G matrix, with following equation:

$$H = ( \begin{matrix}{A}_{11} - {A}_{12} {A}_{22}^{- 1} {A}_{21} + {A}_{12} {A}_{22}^{- 1} {GA}_{22}^{- 1} {A}_{21} & {A}_{12} {A}_{22}^{- 1} G \\ {GA}_{22}^{- 1} {A}_{21} & ( 1 - α ) G + {α A}_{22}\end{matrix} ) ;$$

assigning the individuals to two different groups based on whether the animal individuals in the groups have genotyping information or not, wherein the group with footer “1” represents individuals that only have pedigree information but do not have genomic typing information, the group with footer “2” represents individuals that have both pedigree and genomic typing information, A11 and A22 represent affinity correlation matrices among individuals within group “1” and group “2”, A12 represents the affinity correlation matrices among individuals between the group “1” and the group “2”, A21 is a transpose matrix of A12, and a is a relationship reconciliation percentage for combing G matrix and A22 matrix;

step 2: deriving genetic variance and residual variance from H matrix and phenotype value using HE regression algorithm with following equation:

$$E ( {y}^{T} {A}_{j} y ) = {\sum }_{i = 1}^{n} tr ( {A}_{j} {K}_{i} ) {σ}_{i}^{2} + tr ( {A}_{j} ) {σ}_{n + 1}^{2} ;$$

wherein, y represents phenotypic value vector, σᵢ² is an iₜₕ variance explained by random effects, σₙ₊₁² is residual variance, n is number of random effects in a model, Aⱼ is a symmetric non-negative matrix, Âⱼ is an optimal estimation value of Aⱼ, Âⱼ=H⁻¹KⱼH⁻¹ and H=Σᵢ₌₁ⁿ⁺¹σᵢ²Kᵢ, and Kᵢ and Kⱼ are iₜₕ and jₜₕ additive effect covariate matrices;

step 3: setting the genetic variance and the residual variance from HE regression as prior values of subsequent AI iteration, then deriving the genetic variance and the residual variance using AI iteration algorithm to the convergent standard, and obtaining estimated genetic parameters;

The AI iteration algorithm can be described as parts:

a. Newton-Raphson algorithm:

$${θ}^{( k + 1 )} = {θ}^{( k )} - {( {Hes}^{( k )} )}^{- 1} \frac{\partial L}{\partial θ} {θ}^{( k )} ;$$

wherein θ is genetic parameters to be estimated, k is number of iterations,

$$\frac{\partial L}{\partial θ}$$

is the first derivative of maximum log-likelihood function for each parameter to be estimated, and Hes is a hessian matrix, which is the second derivative of maximum log-likelihood function for each variance;

b. Fisher scoring method, wherein the inverse matrix of Hes is replaced by its expectation matrix F, obtaining

$${θ}^{( k + 1 )} = {θ}^{( k )} - {( {F}^{( k )} )}^{- 1} \frac{\partial L}{\partial θ} {θ}^{( k )} ;$$

the AI matrix can be calculated by

AI=(−Hes+F)/2;

and parameters are estimated with following equation:

$${θ}^{( k + 1 )} = {θ}^{( k )} - {( {AI}^{( k )} )}^{- 1} \frac{\partial L}{\partial θ} {θ}^{( k )} ;$$

step 4: using genetic parameters estimated in step 3 to solve a mixed model equation using Henderson method 3, and getting an estimated breeding value for each individual, wherein the mixed model equation is described as:

$$[ \begin{matrix}{X}^{′} X & {X}^{′} Z \\ {Z}^{′} X & {Z}^{′} Z + {λ K}^{- 1}\end{matrix} ] [ \begin{matrix}\hat{b} \\ \hat{u}\end{matrix} ] = [ \begin{matrix}{X}^{′} y \\ {Z}^{′} y\end{matrix} ] ,$$

V(u)=σᵤ²K, V(e)=σₑ²I, Cov(u,e′)=0, λ=σₑ²/σᵤ², X represents a design matrix corresponding to fixed effects, Z represents a design matrix corresponding to random effects, I represents a unit matrix, K⁻¹ represents an inverse matrix of an affinity relationship matrix, \hat{b} represents an estimated fixed effect vector, and û represents an estimated breeding value vector;

step 5: computing the additive effects of each single nucleotide polymorphism (SNP) marker in genotyping chips with a back solving method, wherein a computing equation can be described as:

$$\hat{a} = \frac{{M}^{′} {K}^{- 1} \hat{u}}{{\sum }_{i = 1}^{m} 2 {p}_{i} {q}_{i}} ;$$

wherein â is an additive effect value vector of SNP markers, m is number of the SNP markers, M′ is the matrix for additive marker covariates, and pᵢ and qᵢ are allele frequency of the iₜₕ SNP genetic markers; and

step 6: when the genotypes of AA, AB, and BB alleles are coded as 0, 1, and 0, respectively, processing a dominant model using the same procedure of step 2 to step 5, and back solving the dominant effect value of each SNP marker.

The technical scheme is adopted to generate the following beneficial effects: according to the fast and stable genomic breeding value evaluating method for animal individuals, provided by the invention, a combined strategy of Haseman-Elston (HE) regression and Average Information (AI) algorithms is used to efficiently obtain a stable estimation of variance components, The HE-AI algorithm based BLUP (namely best linear unbiased prediction) is called “HIBLUP”, HIBLUP makes whole use of pedigree, phenotypic and genotypic information to predict genetic (additive and dominant) effects for each individual animal as well as effect values for each SNP marker, and the most advanced genomic breeding value prediction and variance component estimation algorithm are realized to realize genomic selection.

### BRIEF DESCRIPTION OF DRAWINGS

The sole FIGURE shows a flow chart of a fast and stable genomic breeding value evaluating method for animal individuals according to embodiments of the invention.

### DETAILED DESCRIPTION OF THE PREFERRED EMBODIMENT

The specific implementations of the invention are described in more detail below with reference to the accompanying drawings and embodiments. The following embodiments are intended to illustrate the invention, rather than to limit the scope of the invention.

As shown in the sole FIGURE, the method of the embodiment is described as follows.

According to the fast and stable genomic breeding value evaluating method for animal individuals, HIBLUP is adopted to perform genomic breeding value prediction using phenotype, genotype and pedigree information, and a final output includes estimated genetic value of individuals, additive effect and dominant effect values of each individual, and back solution values of each genetic marker effect used on genotyping chips. The fast and stable genomic breeding value evaluating method for animal individuals specifically includes the following steps:

step 1: performing numeralization on genotypes, coding genotypes of AA, AB and BB as 0, 1, and 2, respectively, constructing a relationship A (affinity correlation IBD) matrix and a G (state correlation IBS) matrix among individuals based on pedigree information using a Henderson tabular method and genomic information using a VanRaden method, respectively, and then constructing a mixture correlation matrix H among the animal individuals according to the information of both an A matrix and a G matrix, with following equation, wherein the matrix includes information from the A matrix and the G matrix, as shown in the below equation:

$$H = ( \begin{matrix}{A}_{11} - {A}_{12} {A}_{22}^{- 1} {A}_{21} + {A}_{12} {A}_{22}^{- 1} {GA}_{22}^{- 1} {A}_{21} & {A}_{12} {A}_{22}^{- 1} G \\ {GA}_{22}^{- 1} {A}_{21} & ( 1 - α ) G + {α A}_{22}\end{matrix} ) ;$$

assigning the individuals to two different groups based on whether the animal individuals in the groups have genotyping information or not, wherein the group with footer “1” represents individuals that only have pedigree information but do not have genomic typing information, the group with footer “2” represents individuals that have both pedigree and genomic typing information, A11 and A22 represent affinity correlation matrices among individuals within group “1” and group “2”, A12 represents the affinity correlation matrices among individuals between the group “1” and the group “2”, A21 is a transpose matrix of A12, and a is a relationship reconciliation percentage for combing G matrix and A22 matrix;

step 2: deriving genetic variance and residual variance from H matrix and phenotype value using HE regression algorithm with following equation:

$$E ( {y}^{T} {A}_{j} y ) = {\sum }_{i = 1}^{n} t r ( {A}_{j} {K}_{i} ) {σ}_{i}^{2} + t r ( {A}_{j} ) {σ}_{n + 1}^{2} ;$$

wherein, y represents phenotypic value vector, σᵢ² is an iₜₕ variance explained by random effects, an σₙ₊₁² is residual variance, n is number of random effects in a model, Aⱼ is a symmetric non-negative matrix, Âⱼ is an optimal estimation value of Aⱼ, Âⱼ=H⁻¹KⱼH⁻¹ and H=Σᵢ₌₁ⁿ⁺¹σᵢ²Kᵢ, and Kᵢ and Kⱼ are iₜₕ and jₜₕ additive effect covariate matrices;

step 3: setting the genetic variance and the residual variance from HE regression as prior values of subsequent AI iteration, then deriving the genetic variance and the residual variance using AI iteration algorithm to the convergent standard, and obtaining estimated genetic parameters;

The AI iteration algorithm can be described as parts:

a. Newton-Raphson algorithm:

$${θ}^{( k + 1 )} = {θ}^{( k )} - {( {Hes}^{( k )} )}^{- 1} \frac{\partial L}{\partial θ} {θ}^{( k )} ;$$

wherein θ is genetic parameters to be estimated, k is number of iterations,

$$\frac{\partial L}{\partial θ}$$

is the first derivative of maximum log-likelihood function for each parameter to be estimated, and Hes is a hessian matrix, which is the second derivative of maximum log-likelihood function for each variance;

b. Fisher scoring method, wherein the inverse matrix of Hes is replaced by its expectation matrix F, obtaining

$${θ}^{( k + 1 )} = {θ}^{( k )} - {( {F}^{( k )} )}^{- 1} \frac{\partial L}{\partial θ} {θ}^{( k )} ;$$

the AI matrix can be calculated by;

AI=(−Hes+F)/2.

and parameters are estimated with following equation:

$${θ}^{( k + 1 )} = {θ}^{( k )} - {( A {I}^{( k )} )}^{- 1} \frac{\partial L}{\partial θ} {θ}^{( k )} ;$$

step 4: using genetic parameters estimated in step 3 to solve a mixed model equation using Henderson method 3, and getting an estimated breeding value for each individual, wherein the mixed model equation is described as:

$$[ \begin{matrix}{X}^{′} X & {X}^{′} Z \\ {Z}^{′} X & {Z}^{′} Z + λ {K}^{- 1}\end{matrix} ] [ \begin{matrix}\hat{b} \\ \overline{u}\end{matrix} ] = [ \begin{matrix}{X}^{′} y \\ {Z}^{′} y\end{matrix} ] ,$$

V(u)=σᵤ²K, V(e)=σₑ²I, Cov(u,e′)=0, λ=σₑ²/σᵤ², X represents a design matrix corresponding to fixed effects, Z represents a design matrix corresponding to random effects, I represents a unit matrix, K⁻¹ represents an inverse matrix of an affinity relationship matrix, \hat{b} represents an estimated fixed effect vector, and û represents an estimated breeding value vector;

step 5: computing the additive effects of each single nucleotide polymorphism (SNP) marker in genotyping chips with a back solving method, wherein a computing equation can be described as:

$$\hat{a} = \frac{{M}^{′} {K}^{- 1} \hat{u}}{{\sum }_{i = 1}^{m} 2 {p}_{i} {q}_{i}} ;$$

wherein â is an additive effect value vector of SNP markers, m is number of the SNP markers, M′ is the matrix for additive marker covariates, and pᵢ and qᵢ are allele frequency of the iₜₕ SNP genetic markers; and

step 6: when the genotypes of AA, AB, and BB alleles are coded as 0, 1, and 0, respectively, processing a dominant model using the same procedure of step 2 to step 5, and back solving the dominant effect value of each SNP marker.

The application of HIBLUP in pig genomic selection can be used to shorten the breeding cycle (time interval), increase the selection accuracy and accelerate genetic gain for traits under selection. The application mainly includes following steps: obtaining genotype data, pedigree data and phenotype data; preparing above described datasets in HIBLUP input data format; running HIBLUP program to get estimated breeding value (EBV) of each individual; computing the selection index using EBVs of multiple traits; and ranking individuals by comprehensive selection index and providing a list of selection candidates.

Finally, it should be noted that the above embodiments are merely intended to describe the technical solutions of the invention, rather than to limit the invention. Although the invention is described in detail with reference to the above embodiments, persons of ordinary skill in the art should understand that they may still make modifications to the technical solutions described in the above embodiments or make equivalent replacements to some or all technical features thereof, without enabling the essence of the technical solutions to depart from the scope defined by the claims of the invention.

What is claimed is:

1. A fast and stable genomic breeding value evaluating method for animal individuals, wherein HIBLUP is adopted to perform genomic breeding value prediction using phenotype, genotype and pedigree information, and a final output includes estimated genetic value of individuals, additive effect and dominant effect values of each individual, and back solution values of each genetic marker effect used on genotyping chips; the method includes the following steps:
step 1: performing numeralization on genotypes, coding genotypes of AA, AB and BB as 0, 1, and 2, respectively, constructing a relationship A (affinity correlation IBD) matrix and a G (state correlation IBS) matrix among individuals based on pedigree information using a Henderson tabular method and genomic information using a VanRaden method, respectively, and then constructing a mixture correlation matrix H among the animal individuals according to information of both an A matrix and a G matrix, with following equation:
$$H = ( \begin{matrix}{A}_{11} - {A}_{12} {A}_{22}^{- 1} {A}_{21} + {A}_{12} {A}_{22}^{- 1} {GA}_{22}^{- 1} {A}_{21} & {A}_{12} {A}_{22}^{- 1} G \\ {GA}_{22}^{- 1} {A}_{21} & ( 1 - α ) G + α {A}_{22}\end{matrix} ) ;$$
assigning the individuals to two different groups based on whether the animal individuals in the groups have genotyping information or not, wherein the group with footer “1” represents individuals that only have pedigree information but do not have genomic typing information, the group with footer “2” represents individuals that have both pedigree and genomic typing information, A₁₁ and A₂₂ represent affinity correlation matrices among individuals within group “1” and group “2”, respectively, A₁₂ represents the affinity correlation matrices among individuals between the group “1” and the group “2”, A₂₁ is a transpose matrix of A12, and α is a relationship reconciliation percentage for combing G matrix and A₂₂ matrix;
step 2: deriving genetic variance and residual variance from H matrix and phenotype value using HE regression algorithm with following equation:
$$E ( {y}^{T} {A}_{j} y ) = {\sum }_{i = 1}^{n} t r ( {A}_{j} {K}_{i} ) {σ}_{i}^{2} + t r ( {A}_{j} ) {σ}_{n + 1}^{2} ;$$
wherein, y represents phenotypic value vector, σᵢ² is an iₜₕ variance explained by random effects, an σₙ₊₁² is residual variance, n is number of random effects in a model, Aⱼ is a symmetric non-negative matrix, Âⱼ is an optimal estimation value of Aⱼ, Âⱼ=H⁻¹KⱼH⁻¹ and H=Σᵢ₌₁ⁿ⁺¹σᵢ²Kᵢ, and Kᵢ and Kⱼ are iₜₕ and jₜₕ additive effect covariate matrices;
step 3: setting the genetic variance and the residual variance from HE regression as prior values of subsequent AI iteration, then deriving the genetic variance and the residual variance using AI iteration algorithm to the convergent standard, and obtaining estimated genetic parameters;
step 4: using genetic parameters estimated in step 3 to solve a mixed model equation using Henderson method 3, and getting an estimated breeding value for each individual, wherein the mixed model equation is described as:
$$[ \begin{matrix}{X}^{′} X & {X}^{′} Z \\ {Z}^{′} X & {Z}^{′} Z + λ {K}^{- 1}\end{matrix} ] [ \begin{matrix}\hat{b} \\ \overline{u}\end{matrix} ] = [ \begin{matrix}{X}^{′} y \\ {Z}^{′} y\end{matrix} ] ,$$
V(u)=σᵤ²K, V(e)=σₑ²I, Cov(u,e′)=0, λ=σₑ²/σᵤ², X represents a design matrix corresponding to fixed effects, Z represents a design matrix corresponding to random effects, I represents a unit matrix, K⁻¹ represents an inverse matrix of an affinity relationship matrix, \hat{b} represents an estimated fixed effect vector, and u represents an estimated breeding value vector;
step 5: computing the additive effects of each single nucleotide polymorphism (SNP) marker in genotyping chips with a back solving method, wherein a computing equation is described as:
$$\hat{a} = \frac{{M}^{′} {K}^{- 1} \hat{u}}{{\sum }_{i = 1}^{m} 2 {p}_{i} {q}_{i}} ;$$
wherein â is an additive effect value vector of SNP markers, m is number of the SNP markers, M′ is a matrix for additive marker covariates, and pᵢ and qᵢ are allele frequency of iₜₕ SNP genetic markers; and
step 6: when genotypes of AA, AB, and BB alleles are coded as 0, 1, and 0, respectively, processing a dominant model using the same procedure of step 2 to step 5, and back solving the dominant effect value of each SNP marker.

2. The method according to claim 1, wherein the AI iteration algorithm in step 3 is described as parts:
a. Newton-Raphson algorithm:
$${θ}^{( k + 1 )} = {θ}^{( k )} - {( {Hes}^{( k )} )}^{- 1} \frac{\partial L}{\partial θ} {θ}^{( k )} ;$$
wherein θ is genetic parameters to be estimated, k is number of iterations,
$$\frac{\partial L}{\partial θ}$$
is the first derivative of maximum log-likelihood function for each parameter to be estimated, and Hes is a hessian matrix, which is the second derivative of maximum log-likelihood function for each variance;
b. Fisher scoring method, wherein the inverse matrix of Hes is replaced by its expectation matrix F, obtaining
$${θ}^{( k + 1 )} = {θ}^{( k )} - {( {F}^{( k )} )}^{- 1} \frac{\partial L}{\partial θ} {θ}^{( k )} ;$$
an AI matrix is calculated by
AI=(−Hes+F)/2;
and parameters are estimated with following equation:
$${θ}^{( k + 1 )} = {θ}^{( k )} - {( A {I}^{( k )} )}^{- 1} \frac{\partial L}{\partial θ} {θ}^{( k )} .$$
